(19) United States
(12) Patent Application Publication
(10) Pub. No.: US 2022/0284832 A1
(43) Pub. Date: Sep. 8, 2022

(54) MODELS AND METHODS TO ESTABLISH PERFUSED VASCULARIZED TISSUES IN THREE-DIMENSIONAL IN VITRO CULTURE

(71) Applicant: Advanced Solutions Life Sciences, LLC, Louisville, KY (US)

(72) Inventors: James B. Hoying, Manchester, NH (US); Michael W. Golway, Louisville, KY (US); Sarah Moss, Manchester, NH (US)

(73) Assignee: Advanced Solutions Life Sciences, LLC, Louisville, KY (US)

(21) Appl. No.: 17/686,878

(22) Filed: Mar. 4, 2022

Related U.S. Application Data
(60) Provisional application No. 63/157,301, filed on Mar. 5, 2021.

(51) Int. Cl. G09B 23/30; C12M 3/00; C12N 5/071

(52) U.S. Cl. CPC G09B 23/306; C12M 21/08; C12N 5/0671; C12N 5/0697

(57) ABSTRACT

Provided herein are 3D tumor angiogenesis models and their methods of preparation and use. In some aspects, the need for identifying whether a potential drug target influences angiogenesis, identifying compounds that modulate angiogenesis, and identifying new drug targets for modulating angiogenesis.

### RELATED APPLICATIONS

This application claims priority to U.S. Provisional Patent Application 63/157,301, filed Mar. 5, 2021, the content of which is hereby incorporated by reference in its entirety.

### TECHNICAL FIELD

The present disclosure relates to field of in vitro tumor models. Specifically, this disclosure relates to vascularized in vitro tumor models and their methods of manufacture and use.

### BACKGROUND

A key feature to tissue viability and function, including tumors, is effective, organotypic vascularization and perfusion of the tissue. Specific to tumors, the tumor microcirculation is integral to tumor growth and is also a route for metastasis. Furthermore, dynamics between the blood-tumor compartments are critical to chemotherapies, radiation therapies, and next-generation immune therapies. An in vitro model in which tumor fragments or vascularized tumor spheroids are integrated with a native microcirculation would be invaluable in understanding better tumor biology and tumor pathology, as well as modeling more completely the in vivo tumor environment in drug screens and therapy development efforts.

A need exists for improved models and methods that approximate the complexity of native vascularization and may be used to gain insight into tumor biology and as a model to study disruption thereto for improved therapeutic outcomes.

### SUMMARY

The following summary is provided to facilitate an understanding of some of the innovative features unique to the present disclosure and is not intended to be a full description. A full appreciation of the various aspects of the disclosure can be gained by taking the entire specification, claims, drawings, and abstract as a whole.

Provided herein are methods of preparing three-dimensional (3D) models that allow for vascularization of tumor spheroids and/or organoids to study the mechanisms by which tumors interact with the vasculature, as well as for the study of agents and/or mechanisms by which tumor vascularization may be disrupted or ceased.

In one aspect, the present disclosure concerns a three-dimensional (3D) tumor model that includes tumor cells; and isolated microvessel fragments or a microvasculature developed therefrom. The isolated microvessel fragments or the microvasculature may be embedded within a polymerized medium comprised of extracellular matrix.

In some aspects, the extracellular matrix may include at least one of collagen I, collagen II, collagen III, collagen IV, fibrin, Matrigel, laminin, nidogen, perlecan sulfated glycolipids, glycoproteins and/or proteoglycans.

In some aspects, the tumor cells are alone or part of a tumor organoid, a tumor spheroid, or a pre-vascularized tumor fragment.

In some aspects, the model may further include a first and a second channel. In some aspects, the two channels are parallel. In some aspects, the first and second channels are embedded within the polymerized medium. In some aspects, the isolated microvessel fragments or the microvasculature developed therefrom are in a space between the first and second channels. In some aspects each of the first and second channels includes an inlet end and an outlet end and further wherein a fluid source is operably connected to each inlet end. In some aspects, each outlet end is operably connected to an outlet reservoir.

In some aspects the model may further include an at least partial obstruction at the outlet end of the first channel to provide a pre-load pressure. In some aspects, the at least partial obstruction includes a collagen plug. In some aspects, the outlet reservoir is operably connected to at least the inlet end of the second channel.

In further aspects, one or more extracellular matrix proteins and/or structures are in contact with the tumor cells. In certain aspects, the one or more extracellular matrix proteins and/or structures comprise basement membrane proteins and/or structures.

In some aspects, the present disclosure concerns a method for preparing a vascularized 3D tumor model through: providing isolated microvessel fragments to a space between two channels embedded within a polymerized medium; and providing tumor cells on or embedded within the polymerized medium.

In some aspects, the method relates to the tumor cells that are part of a tumor organoid, a tumor spheroid, or a pre-vascularized tumor fragment.

In some aspects, a fluid media is perfused through the inlet of one channel to an outlet reservoir and back through an inlet of the second channel. In further aspects, the fluid media is perfused at a rate of about 20 μL/hour.

In some aspects, the tumor cells are in contact with a one or more extracellular matrix proteins and/or structures.

In some aspects, the method also includes providing isolated endothelial cells to the fluid media. In certain aspects, at least one outlet end is at least partially obscured to create a pre-load pressure in the channel, such as through a collagen plug.

In some aspects, the present disclosure concerns a method for preparing a vascularized 3D tumor model through: providing isolated microvessel fragments to a space between a first channel and a second channel embedded within a polymerized medium; incubating the isolated microvessel fragments within the polymerized medium for a period of four to six days or until angiogenesis is observed; perfusing a fluid media from an inlet reservoir through the first channel to an outlet reservoir, wherein the outlet reservoir is operably connected to the second channel such that the perfused fluid media can traverse the second channel, wherein the fluid media is perfused for about three to five days or until the isolated microvessel fragments have visibly inosculated; at least partially obscuring the first channel at the outlet to the outlet reservoir to provide an increased pre-load pressure; re-initiating perfusion of the fluid media; and providing tumor cells on or embedded within the polymerized medium.

In some aspects, the method further includes providing isolated endothelial cells to at least the first channel. In some aspects, the tumor cells are provided prior to incubation of the isolated microvessel fragments. In other aspects, the tumor cells are provided following the re-initiation of perfusion of the fluid media.

In some aspects, the fluid media is perfused at a rate of about 10 to 1000 μL/hr. In further aspects, the increased pre-load pressure is of about 0.5 mm of Hg to 160 mm of Hg.

In some aspects, the present disclosure concerns a method for preparing a vascularized 3D model through: providing isolated microvessel fragments to a space between a first channel and a second channel embedded within a polymerized medium; incubating the isolated microvessel fragments within the polymerized medium for a period of four to six days or until angiogenesis is observed; perfusing a fluid media from an inlet reservoir through the first channel to an outlet reservoir, wherein the outlet reservoir is operably connected to the second channel such that the perfused fluid media can traverse the second channel, wherein the fluid media is perfused for about three to five days or until the isolated microvessel fragments have visibly inosculated; at least partially obscuring the first channel at the outlet to the outlet reservoir to provide an increased pressure; providing isolated endothelial cells to the first and second channels; and re-initiating perfusion of the fluid media.

In some aspects, the method further includes providing isolated endothelial cells to at least the first channel. In some aspects the tumor cells are provided prior to incubation of the isolated microvessel fragments. In other aspects, the tumor cells are provided following the re-initiation of perfusion of the fluid media.

In some aspects, the fluid media is perfused at a rate of about 10 to 1000 μL/hr. In further aspects, the increased pre-load pressure is of about 0.5 mm of Hg to 160 mm of Hg.

In some aspects, the present disclosure concerns a 3D angiogenesis model that includes isolated microvessel fragments or a microvasculature developed therefrom between two parallel channels embedded within a polymerized medium. In some aspects, each channel includes an inlet end and an outlet end, each inlet end being operably connected to a fluid media source. In some aspects, at least one outlet end is operably linked to the inlet end of a different channel. In some aspects, the fluid media is actively pumped into at least one channel to allow for interstitial flow-conditioning.

In some aspects, the model further includes tumor cells on or embedded within the polymerized medium. In some aspects, the model further includes an at least partial obstruction at the outlet end of the first channel to provide a pre-load pressure, such as through a collagen plug.

### BRIEF DESCRIPTION OF THE DRAWINGS

The aspects set forth in the drawings are illustrative and exemplary in nature and not intended to limit the subject matter defined by the claims. The following detailed description of the illustrative aspects can be understood when read in conjunction with the following drawings, where like structure is indicated with like reference numerals and in which:

FIG. 1 shows an overview of isolated microvessel fragment capability in 3D cell culture. A shows phase images of isolated human microvessel fragments (MV, open arrows). B shows a 3D matrix culture undergoing angiogenesis (closed arrows=neovessels, *=parent MVs). C shows isolated MVs are intact and comprised of a variety of cell types. D shows the isolated MVs with fluorescent staining.

FIG. 2 illustrates neovascular network formation and engraftment. (A) depicts three still images (left panel, center panel, and middle panel) from a time lapse video of an inosculation event (closed arrow) between 2 neovessels (open arrow) during angiogenesis in 3D stromal collagen. (B, C) depict phase images of a neovascular network and a shaded volume rendering of a network showing continuity. (D) depicts resulting microcirculation following transplantation of a neovasculature.

FIG. 3 depicts example images of an endothelial cell (EC)-lined channel surrounded by growing neovessels forming a network (black arrow heads in phase image) adjacent to the channel walls (open arrows). Neovessels inosculate with the ECs of the channel enabling perfusion of beads (right panels) as shown by still images from real-time video showing two beads moving through neovessels (upper left). Dashed lines indicate flow paths. Stationary beads marked for positional reference.

FIG. 4 is a schematic highlighting the strategy for incorporating for tumor cells or spheroids into the model for in vitro perfusion.

FIG. 5 shows phase (left panel) and fluorescence (right panel) images of pre-vascularized tumor organoids growing in stroma containing growing microvessels.

FIG. 6 shows one aspect of the perfused model (200). Two channels (210, 220) are provided within a polymerized medium or matrix (230). One end or an inlet of one channel (210) is operably connected to an inlet reservoir (240) wherein pressure and/or a pump can cause a fluid media to flow and perfuse the channel (210) and exit from its other end or outlet and fill into an outlet reservoir (250). The outlet reservoir (250) is also arranged such that it is in open communication with an end or inlet of the second channel (220). Accordingly, as fluid media fills into or out of the outlet reservoir, sufficient pressure is provided that allows for the second channel (220) to be effectively perfused and empty from its other end or outlet into a second outlet reservoir (260). The microvessel fragments (270) are placed between the two channels (210, 220) and accordingly as the microvessel fragments (270) inosculate with the channels, the newly formed microvasculature is operably connected to the now perfused two channels (210, 220) thereby providing for intravascular perfusion of the microvasculature itself and thus the perfused tissue model (200).

FIG. 7 shows an overhead cartoon of the three phases for providing inosculated and perfused microvasculature from microvessel fragments with a profile cartoon next to each stage illustrating the progress of the microvasculature development.

FIG. 8 shows H&E staining (top panel) and fluorescent staining (bottom panel) of a cross section of a perfused vessel within the 3D model.

FIG. 9 depicts schematics of the two model configurations for a perfusion model. In Model 1, tumor cells (10) are established on top of a 3D collagen matrix/polymerized medium (20) surrounding a microcirculation (30) connected to perfused (40) channels. In this model, basement membrane proteins can also be coated onto the matrix prior to adding the tumor cells. This configuration models EMT and tumor invasion. In Model 2, prevascularized tumor spheroids (50) are integrated into the microcirculation (30) such that the spheroid vasculature and the stromal microcirculation have inosculated. This configuration models native tumor biology, cancer therapies, and metastasis.

FIG. 10 shows vascularization of a tumor with the model as set forth herein. The top panel shows a microscopic image of the tumor in the 3D culture. The bottom panel shows vasculature from the model (arrows) entering into the tumor mass (circle).

FIG. 11 shows a phase microscopy image of vessel in growth into the bulk tumor (arrows).

### DETAILED DESCRIPTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes or compositions are described as an order of individual steps or using specific materials, it is appreciated that steps or materials may be interchangeable such that the description of the invention may include multiple parts or steps arranged in many ways as is readily appreciated by one of skill in the art.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Wild, D., The Immunoassay Handbook, 3rd Ed., Elsevier Science, 2005; Gosling, J. P., Immunoassays: A Practical Approach, Practical Approach Series, Oxford University Press, 2005; Antibody Engineering, Kontermann, R. and Dübel, S. (Eds.), Springer, 2001; Harlow, E. and Lane, D., Antibodies: A Laboratory Manual, Cold Spring Harbor Laboratory Press, 1988; Ausubel, F. et al., (Eds.), Short Protocols in Molecular Biology, Wiley, 2002; J. D. Pound (Ed.) Immunochemical Protocols, Methods in Molecular Biology, Humana Press; 2nd ed., 1998; B. K. C. Lo (Ed.), Antibody Engineering: Methods and Protocols, Methods in Molecular Biology, Humana Press, 2003; and Kohler, G. and Milstein, C., Nature, 256:495-497 (1975); the contents of each of which are incorporated herein by reference.

In some aspects, the present disclosure concerns a 3D cell culture of a pre-vascularized tumor fragment or tumor spheroid or tumor organoid and a microvasculature within a 3D polymerized medium or matrix. In some aspects, the microvasculature is perfused. In some aspects, the microvasculature is part of a perfusion model. In further aspects, methods of assembling and observing or testing the assembled cell culture are also provided.

In some aspects, the present disclosure concerns providing a spheroid and/or an organoid to an in vitro cellular three dimensional (3D) matrix. Tissue organoids are useful tools for many different applications, including modeling diseases or high throughput screening of potential therapeutics. Organoids are three-dimensional, self-organized constructs comprising different types of organ-specific cells that are assembled into aggregates or derived within a tissue construct, ranging from tens of microns to several millimeters in diameter. The terms “organoid” and “spheroid” are often used interchangeably in the art. However, it should be understood that “spheroid” typically refers to a three-dimensional aggregate of cells, which may be comprised of a single cell type or of multiple cell types. Spheroids are commonly used to culture or differentiate stem cells, which require a 3D structure, but which do not necessarily mimic the complexity and function of a tissue. Organoids are typically more complex, containing intricate connections between multiple cell types and matrix components often compartmentalized and functioning as a tissue, thereby enabling the investigation of cellular behavior in a biologically relevant tissue environment. Numerous tissue types have been modeled as organoids, including adipose, brain, liver, kidney, and and the like, said tissues being fabricated using a number of different aggregation methods known in the art.

In some aspects, the present disclosure concerns preparing or isolating a spheroid or organoid. In some aspects, the spheroid or organoid may be prepared by isolating or obtaining at least one cell type. In some aspects, the present disclosure concerns preparing an organoid or spheroid and providing such to a 3D cell culture. In some aspects, the organoid or spheroid is derived from tumor cells or a tumor mass. For example, cells can be excised from a subject and utilized immediately or optionally first treated and/or cultured to remove unwanted extracellular matrix material or tissue and/or enzyme treated, such as with trypsin, to loosen cell-cell associations. In some aspects, the methods may include isolating or obtaining at least one tumor cell type, such as a cancerous or pre-cancerous cell from the breast, lung, liver, kidney, epidermis, colon, pancreas, neurological system, brain, lymphatic system, bone, muscle, prostate, bladder, intestine, ovary, or testes. Tumor cells may be isolated or may be part of an isolate from extracted or resected tumor tissue. In some aspects, tumor cells may include established in vitro cell culture cells that are known to be tumorigenic or pre-cancerous. In some aspects, the tumor spheroid and/or organoid can be prepared by co-culturing a tumor cell line with at least a second cell line that can be cancerous or non-cancerous. In some aspects, the tumor spheroid or tumor organoid can be prepared by obtaining at least a portion of an excised tumor and partially digesting to obtain a cluster of at least one cell type. In some aspects, a pre-vascularized tumor fragment may be utilized. It will be appreciated that in some aspects, a partial digest may include disruption of cell-cell interactions such that the associations between cells are loosened. In some aspects, loosening cell-cell interactions may provide for easier vascularization when grown in 3D culture. In some aspects, additional cells may be added into the spheroid or organoid to allow for the desired cell-cell interactions and/or a closer approximation to a particular organ or tumor type. In some aspects, one or more cells may be pretreated, such as with a tumorigenic compound, an initiating compound, an experimental compound, a chemotherapeutic, or other compound. In some aspects, a tumor cell or combination of tumor cells may be cultured together with one or more further cells that may include stem cells, progenitor cells, mesenchymal cells, endothelial cells, perivascular cells, fibroblasts, endothelial lineage cells, or combinations thereof. In some aspects, the cells may include one or more programmed cells. It will be appreciated that cells utilized for the spheroid or organoid can be derived from any cell type, as well as combined with any cell type. It will further be appreciated that while any tumor cell type may be included or selected, in some aspects as the methods herein allow for assessment of vascularization of spheroids and/or organoids, tumors that rely on creating vascular networks may in some aspects be particularly useful.

In some aspects, the methods of the present disclosure concern preparing a tumor spheroid or tumor organoid or pre-vascularized tumor fragment prior to introduction into a 3D cell culture system. In some aspects, the pre-vascularized tumor fragment or tumor spheroid or tumor organoid may be pre-cultured to allow the cells therein to adjust to other cell types and/or cell culture conditions and/or media. In some aspects, the tumor spheroid or tumor organoid may be pre-vascularized. In some aspects, the cells may be pre-treated with pro-angiogenic factors. In some aspects, the cells may be pre-cultured in a 3D matrix prior to vascularization thereof. In some aspects, tumor cells may be co-cultured with micro vessels to form a spheroid or organoid and/or to pre-vascularize the tumor cells. The spheroid or organoid may then be introduced to the 3D polymerized medium or matrix.

In some aspects, the present disclosure concerns placing a tumor spheroid or tumor organoid or pre-vascularized tumor fragment in a 3D in vitro culture. In some aspects the tumor spheroid or tumor organoid or pre-vascularized tumor fragment is placed on or embedded within a 3D polymerized medium or matrix. In some aspects, the tumor spheroid or tumor organoid or pre-vascularized tumor fragment is placed on or mixed with extracellular matrix proteins and/or structures, including basement membrane proteins and/or basement membrane structures, such as collagen IV, laminin, nidogen, perlecan sulfated glycolipids, as well as glycoproteins and/or proteoglycans. In some aspects, an organoid is placed in the 3D culture. Organoids can be advantageously compared to other scaffold-based engineered tissues because cells are in a dense 3D environment with numerous direct cell-cell and cell-matrix contacts, as they would be in the native tissue environment. In some aspects, 3D cultures preserve cell phenotype and function more effectively than 2D cultures. For example, certain primary cell types, including osteoblasts, smooth muscle cells, and hepatocytes rapidly lose their phenotypes in 2D culture, but are less prone to losing their phenotypes in 3D culture environments.

In some aspects, the methods of the present disclosure concern providing a vasculature to tumor spheroid or tumor organoid or pre-vascularized tumor fragment in a 3D culture. In some aspects, the tumor spheroid and/or tumor organoid and/or pre-vascularized tumor fragment is provided to an established vasculature or microvasculature. In other aspects, the tumor spheroid and/or tumor organoid and/or pre-vascularized tumor fragment is provided to a developing or growing vasculature or microvasculature. In further aspects, the tumor spheroid and/or tumor organoid and/or pre-vascularized tumor fragment is provided to a 3D culture simultaneously or contemporaneously with vasculature precursors or microvessel fragments. It will be appreciated that interacting tumor cells within the tumor spheroids and/or tumor organoids and/or pre-vascularized tumor fragment with differing levels of vasculature development will allow for assessing different aspects of how a tumor can adopt or co-opt the vasculature within a subject and provide necessary perfusion thereto. Native tissues contain a complex, hierarchical network of perfused blood vessels supplying nutrients to and removing waste from tissues too thick or dense to allow for adequate diffusion. The vasculature is also essential for modulating movement of cells between different tissue compartments and serves as a blood-tissue interface. Furthermore, the variety of cell types comprising the vessel wall, including the perivascular niche, such as endothelial cells (EC), mesenchymal stem cells (MSCs), macrophages, pericytes, immune cells, and other progenitor cells, are communicating with the other cells and matrix of the tissue. This creates a dynamic tissue environment determining proper tissue behavior and function. Tumors similarly require a dynamic environment to survive and grow. In order for tumors to sustain growth, the vasculature is co-opted and incorporated therein in order to provide essential nutrients and oxygen to the tumor cells throughout the depth of the tumor mass. Moreover, the larger the tumor, the more perfusion is required. Therefore, a platform and methodology is provided herein that allows for the study of both how tumor cells can achieve perfusion, as well as study how test agents or compounds can disrupt the mechanism(s) by which tumor cells develop their own vasculature.

In some aspects, the present disclosure concerns providing vascular precursors or microvessel fragments (MVs) to a 3D cell culture. In some aspects, the 3D cell culture includes factors that allow for the vasculature precursors or MVs to grow and create a microvasculature. In some aspects, the 3D cell culture includes at least collagen, such as collagen I, II, III and/or IV. In some aspects, the 3D cell culture includes a medium of a polymerized gel from a pre-polymerization solution in a vessel. In some aspects, the pre-polymerization solution is from a collagen solution, a fibrin solution, a Matrigel solution, a laminin solution, or combinations thereof, and then permitting and/or initiating polymerization to form the gel. In some aspects, the solution for polymerization may include at least one of collagen I, collagen II, collagen III, collagen IV, fibrin, Matrigel, laminin, nidogen, perlecan sulfated glycolipids, glycoproteins and/or proteoglycans. In some aspects, the polymerized cell culture media may further include additional cell culture co-factors such as albumin, antibiotics, growth factors, cytokines, salts, sodium, potassium, calcium, phosphates, chlorides, and the like. In some aspects, the 3D cell culture further includes at least one channel on or embedded in a polymerized medium or matrix. In some aspects, the channel is connected to a reservoir or source such that a fluid media can flow through the channel. In some aspects, there are two or more channels. In some aspects, the channels are parallel. In some aspects, each channel is connected to either the same or independent reservoirs or sources of fluid media. In further aspects, a further reservoir is included to collect fluid media from the outlet of each channel. In some aspects, the flow of media may be arranged such that the outlet from one channel provides fluid media into an inlet of another channel. In some aspects, fluid flow or perfusion within the channels allows for MVs to inosculate.

In some aspects, the 3D cell culture includes the introduction of MVs to the medium of the 3D cell culture. The MVs can be provided pre or post polymerization, or during polymerization. In some aspects, the MVs are added to the 3D culture in a space between two channels on or embedded within the 3D polymerized medium or matrix. In some aspects, the MVs are added to the 3D culture prior to a tumor spheroid or tumor organoid or pre-vascularized tumor fragment. In other aspects, a tumor spheroid or tumor organoid or pre-vascularized tumor fragment may be suspended or placed on the 3D cell culture medium prior to introduction of MVs to establish a microvasculature therein. In other aspects, a tumor spheroid or tumor organoid or pre-vascularized tumor fragment may be introduced into the 3D cell culture medium simultaneously or contemporaneously with the MVs. It will be appreciated that providing the tumor spheroid or tumor organoid or pre-vascularized tumor fragment to the 3D cell culture medium at varying time points with regard to the presence of an established or developing microvasculature can allow for studying different aspects of how a tumor cell may work/interact/signal for the microvasculature to develop within the spheroid or organoid space and provide perfusion therein. There are two primary ways vascularization occurs in nature, vasculogenesis and angiogenesis. Vasculogenesis occurs when individual vascular cells “self-assemble” into a neovascular network. Angiogenesis, the primary means of vascularization in the adult, occurs when new vessels sprout from existing vessels. New sprouts will elongate, migrate, and inosculate with other vessels to form a neovascular network. In all neovessel networks, maturation and remodeling occurs, whereby vessels may prune or change morphology in response to changing hemostatic pressure, intravascular communication, and metabolic needs of the tissue. Neovessel maturation may occur throughout the processes of angiogenesis and general remodeling as different regions of the neovasculature receive relevant stimuli. Additionally, during this dynamic remodeling, vessels are stabilized by pericytes and other perivascular cells, perfusion is established, and vessels adapt distinct arteriolar, venular, or capillary phenotype, all in a dynamic, co-dependent process. Most strategies for establishing native vasculatures in organoids rely on some combination of vasculogenesis and angiogenesis to form a neovascular network within spheroids or organoids.

In some aspects, the 3D model is established through the introduction of microvessel fragments in a 3D polymerized medium or matrix. Intact isolated microvessel fragments (MVs) that are obtained from living subjects retain their native structure and multi-cellular composition when cultured in a 3D matrix and will undergo sprouting angiogenesis similarly to vessels in the native, in vivo environment. In some aspects, the MVs are isolated from human adipose aspirates. In some aspects, the MVs develop within the 3D cell culture medium or matrix to a mature microvessel structure with a preserved lumen, an intact basement membrane, an endothelial cell monolayer and at least one layer of perivascular cells.

The application of an MV system into an informative in vitro angiogenesis assay compatible with existing assessment approaches (e.g., high content analysis) provides a more biologically relevant assay. Because angiogenesis, vascular remodeling, and vascular stability depend not only on the endothelial cell, but also proper vessel architecture, mature matrix elements, and a spectrum of perivascular cells, an angiogenesis technology has been developed utilizing freshly isolated microvessel fragments from adipose. Importantly, these isolated microvessel fragments contain all vascular cells types, maintained in the native microvessel structure. When the constructs are placed in 3D polymerized medium or matrix cultures, the individual microvessel fragments spontaneously sprout and grow, forming neovessels which will eventually fill the gel. When implanted as part of a tissue construct, the microvessel fragments recapitulate tissue neovascularization and form stable, perfused, hierarchical microvascular networks. In a variety of applications, isolated microvessel fragments have been explored in the investigation of stromal cell and vascular precursor dynamics, angiogenesis-tissue biomechanics, imaging modalities to assess neovascular behavior, post-angiogenesis microvascular maturation and patterning, characterize angiogenic factors, and evaluate microvascular instability. Additionally, given the rapid means of vascularization, the isolated microvessel fragments have been explored in pre-clinical studies of tissue implants. It is this isolated microvessel system can be used in the generation of in vitro neovasculatures of the Vascularized in vitro perfusion module (VIPM™).

In some aspects, the methods of the present disclosure concern establishing perfusion to the microvasculature within the 3D polymerized medium or matrix. In some aspects, perfusion can be established by providing channels for inosculation by the MVs following their addition within the 3D polymerized medium or matrix. In some aspects, the channels number at least two and are parallel. In some aspects, the channels are separated by a distance of about 2 to 15 mm, including 3, 4, 5, 6, 7, 8, 9, 10, 11, 12, 13, and 14 mm. In some aspects, the 3D matrix includes two parallel channels separated by about 5 mm. In some aspects, the channels are connected to inlet and outlet media reservoirs to allow for fluid media to be pumped in between. In some aspects, the channels are a continuous void within the polymerized medium or matrix such that the polymerized medium or matrix forms the walls of each channel. In some aspects, the channels may be formed by removal of a solid channel mold after polymerization. In other aspects, a channel may be formed by inserting a solid walled structure into the liquid medium prior to polymerization or completion thereof and withdrawal of the solid walled structure after polymerization. In addition to cast-molding the channels, other techniques may similarly provide a channel, such as by boring. It will be appreciated, however, that a mold may provide an even channel width and direction. In some aspects, the channel has a diameter or cross-sectional width of between 10 μm and 1000 μm. In some aspects, each channel has a diameter of about 200 μm.

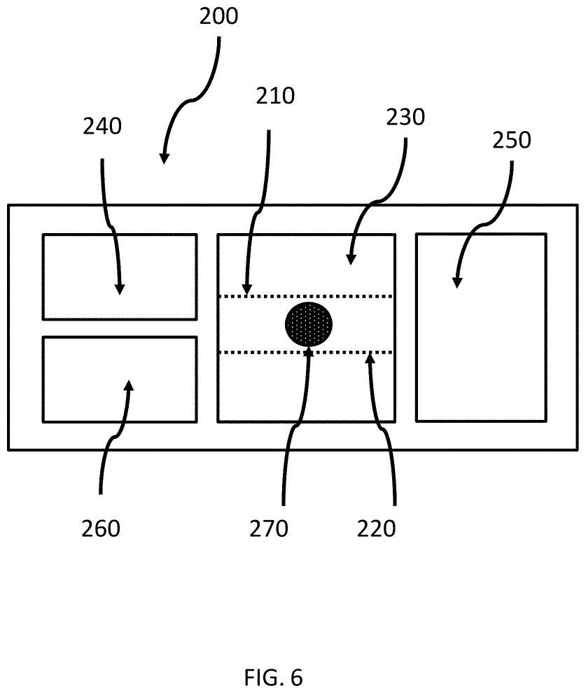

In some aspects, the present disclosure provides for a perfused model. Looking at FIG. 6, one aspect of the perfused model (200) is provided. Two channels (210, 220) are provided within a polymerized medium or matrix (230). One end or an inlet of one channel (210) (inlet channel) is operably connected to an inlet reservoir (240) wherein pressure and/or a pump can cause a fluid media to flow and perfuse the channel (210) and exit from its other end or outlet and fill into an outlet reservoir (250). The outlet reservoir (250) is also arranged such that it is in open communication with an end or inlet of the second channel (220) (outlet channel). Accordingly, as fluid media fills in the outlet reservoir, sufficient pressure is provided that allows for the second channel (220) to be effectively perfused and empty from its other end or outlet into a second outlet reservoir (260). The microvessel fragments (270) are placed between the two channels (210, 220) and accordingly as the microvessel fragments (270) inosculate, the newly formed microvasculature is operably connected to the now perfused two channels (210, 220) thereby providing for perfusion to the microvasculature itself and thus the perfused model (200).

In some aspects, the perfused module can be established by utilization of at least three distinct stages. In some aspects, the first stage includes a seeding step, wherein MVs are seeded between channels. This step may then allow for sprouting and early neovessel elongation from the seeded MVs in a non-perfused and static phase. In some aspects, one skilled in the art can proceed on from the seeding or static step after observing angiogenesis or the beginnings thereof. Such signs may include neovessel sprouting or neovessel elongation. In certain aspects, neovessel elongation should be observed prior to proceeding.

After the MVs demonstrate signs of angiogenesis, media can be perfused through the channels for a second stage of interstitial flow-conditioning. In some aspects, over a period of between about 2-7 days, including 4, 5, and 6 days, fluid media is perfused into and withdrawn from inlet and outlet reservoirs, respectively that allows the fluid media to traverse the inlet channel to a shared reservoir and then be withdrawn back out via the outlet channel. In some aspects, the fluid media is perfused at a steady rate. For example, as set forth in the examples herein, the fluid media can be perfused at about 10-5000 μl/hr, including about 15, 20, 25, 30, 35, 40, 45, 50, 55, 60, 65, 70, 75, 80, 85, 90, 95, 100, 200, 300, 400, 500, 600, 700, 800, and 900 μl/hr. This second stage of interstitial flow-conditioning allows for neovascularity to expand within the 3D polymerized medium or matrix and for neovessels to grow toward both inlet and outlet channels while also inosculating with each other to form interconnected networks.

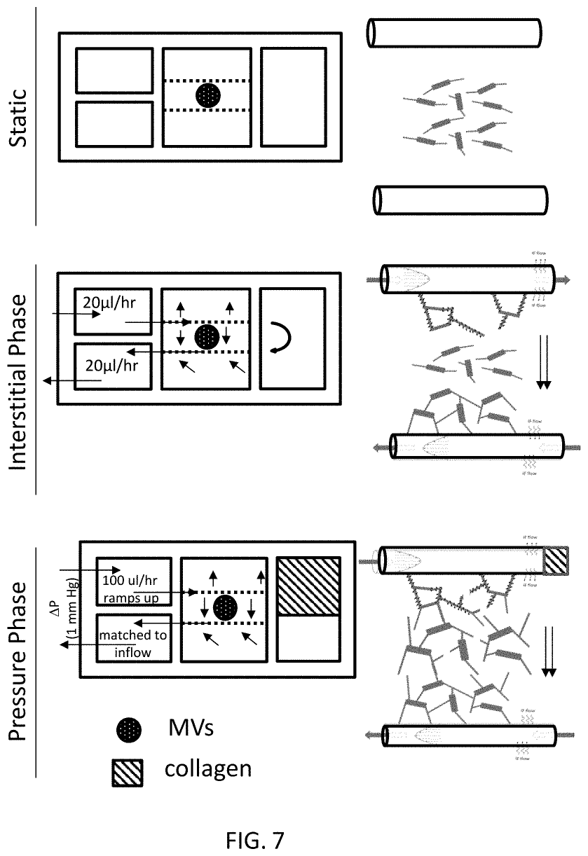

The third phase can then occur by introducing a pre-load pressure to one channel, for example the inlet channel. In some aspects, the inlet channel can be effectively blocked by filling the associated reservoir with collagen while maintaining the filling of the inlet reservoir. In some aspects, the outlet of the inlet channel is partially obscured to provide a preload pressure, In some aspect, the outlet is obscured to provide a pre-load pressure of between about 0.5 and about 160 mm of Hg (mercury), including 1, 2, 3, 4, 5, 6, 7, 8, 9, 10, 20, 30, 40, 50, 60, 70, 80, and 90 mm of Hg. Blocking the channel can then result in an increase in pre-load pressure of about 1 mm Hg to the inlet while fluid media continues to flow through both channels. Endothelial cells can then be introduced into the interior of the channels to allow for the cells to line the channel walls prior to then re-initiating flow to reduce the extent of fluid flow through the interstitium of the collagen. In some aspects, the re-initiated flow is at a rate of about 100 μL/hr. In some aspects, the three stages are arranged as set forth in FIG. 7.

In some aspects, the present disclosure concerns methods of establishing a perfusion model within a 3D polymerized medium or matrix. As described herein, providing or seeding MVs between two channels within the 3D polymerized medium or matrix and continuing to an interstitial flow-conditioning phase for a period of about 2-7 days and then establishing a pre-load pressure therein and providing endothelial cells allows for the MVs to inosculate and create an interconnected vascular network within the 3D polymerized medium or matrix.

In some aspects, the present disclosure concerns methods of studying the development to the inosculated interconnected network from the seeded MVs. In some aspects, the present disclosure concerns providing a pre-vascularized tumor fragment or a tumor spheroid or a tumor organoid to the perfused model. While some aspects of the present disclosure concern the ability of tumor cells to co-opt the developing or developed vasculature of the perfused model, it will be appreciated that non-tumor related spheroids, organoids, tissue fragments, or cells can be similarly utilized either alone or in conjunction with the tumor cells.

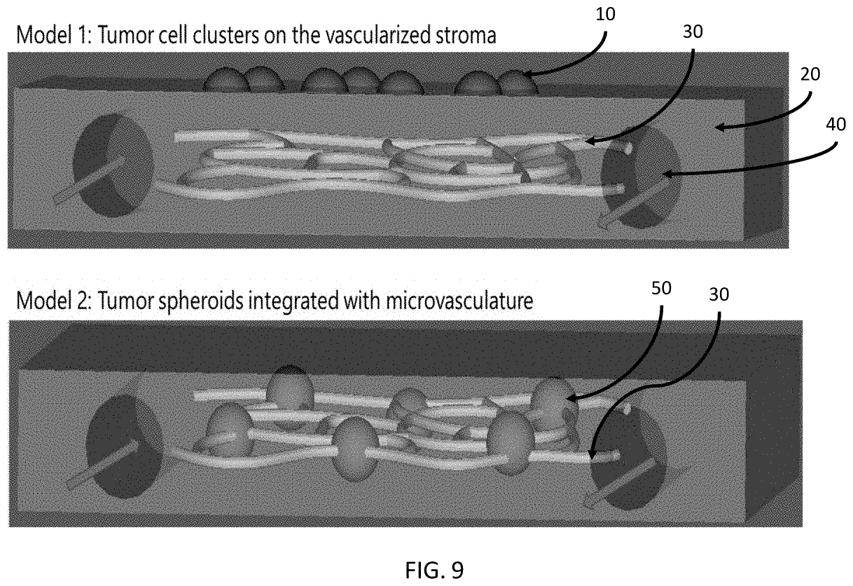

In some aspects, the present disclosure concerns methods of providing a pre-vascularized tumor fragment or a tumor spheroid or a tumor organoid to the perfused model prior to the seeding or static phase. In some aspects, the present disclosure concerns providing a pre-vascularized tumor fragment or a tumor spheroid or a tumor organoid to the perfused model between the seeding/static phase and the interstitial flow-conditioning phase of the perfusion model. In some aspects, the present disclosure concerns providing a pre-vascularized tumor fragment or a tumor spheroid or a tumor organoid to the perfused model between the interstitial flow-condition phase and the pre-load pressure phase of the perfusion model. In some aspects, the present disclosure concerns providing a pre-vascularized tumor fragment or a tumor spheroid or a tumor organoid to the perfused model prior to adding the endothelial cells during the pre-load pressure phase. In some aspects, the present disclosure concerns providing a pre-vascularized tumor fragment or a tumor spheroid or a tumor organoid to the perfused model after completion of the pre-load pressure phase. In further aspects, a pre-vascularized tumor fragment or a tumor spheroid or a tumor organoid to the perfused model at multiple points throughout the three phases. For example, FIG. 9 depicts schematics of the two potential configurations. In the upper model, tumor cells (10) are provided on top of a polymerized medium or matrix (20) surrounding a microcirculation (30) connected to perfused (40) channels. In this model, basement membrane proteins can also be coated onto the matrix prior to adding the tumor cells. This configuration models EMT and tumor invasion. In the lower model, prevascularized tumor spheroids (50) are integrated into the microcirculation (30) such that the spheroid vasculature and the stromal microcirculation are inosculated. This configuration models native tumor biology, cancer therapies, and metastasis.

In some aspects, the present disclosure concerns utilization of the perfusion model to identify agents or to study agents or mechanisms whereby agents affect vascularization within the perfusion model. Test agents may include small molecules, chemical compounds or combinations thereof, nucleotides, peptides, proteins, growth factors, pharmaceutical compounds, lipids, carbohydrates, combinations thereof or similar. Agents can be applied prior to seeding and/or during the static phase and/or during the interstitial flow-conditioning phase and/or during the pre-load phase and/or post pre-load phase. It will be appreciated that through the co-application of sufficient control perfusion arrangements, those skilled in the art can identify significant information as to how a test agent or compound may enhance or disrupt or generally affect the development of the microvasculature from the MVs, as well as how the MVs inosculate and form the network. In further aspects, cells, spheroids, and/or organoids can be included within the assays to determine both their natural interaction with the perfusion model and developing/developed microvasculature as well as how an applied or administered test agent may disrupt or enhance or generally affect such a relationship.

In some aspects, the perfusion model is provided with a pre-vascularized tumor fragment or a tumor spheroid or a tumor organoid and a test agent. As identified herein, the order of introduction of each can be varied and may be dependent on the user's primary point of focus. It will be however appreciated that the application of an agent to assess or measure disruption of vascularization within a tumor will be most clinically relevant where the inosculated microvasculature is allowed to be established followed by introduction of the pre-vascularized tumor fragment or tumor spheroid or tumor organoid and then followed by application of the test agent. It will be appreciated that in some aspects, a user may want an applied pre-vascularized tumor fragment or tumor spheroid or tumor organoid to first integrate or initiate co-opting the microvasculature prior to application of a test agent. For example, in certain aspects, a user may prefer to allow for a period of days or weeks to pass prior to administration of a test agent.

In some aspects, the methods of the present disclosure may include observing or assaying cells from the perfusion model. In some aspects, the methods may include observing or assaying the amount of vasculature adopted of co-opted by the pre-vascularized tumor fragment or tumor spheroid or tumor organoid. In some aspects, the methods may include observing or measuring tumor cell growth and/or number. For example, adopting or co-opting a nearby vasculature allows for a tumor to become perfused and increase chances of survival as well as allow for growth to be less restrained. Monitoring or measuring tumor cell number or tumor mass allows for an understanding of the health of the tumor cells. Such may provide further information as to the level of effect that a test agent is providing. In some aspects, the methods of the present disclosure concern assessing and/or observing angiogenic changes, including the development of vascularization within the tumor cell, pre-vascularized fragment, spheroids and/or organoids. Angiogenic changes and/or growth can be determined and/or measured using measuring devices and/or calculating devices to determine that amount of change and/or growth. In some aspects, the rate of change over a period of time may be observed and/or calculated. One aspect of the present disclosure concerns artificial intelligence for microvessel quantification: In one aspect, assessments of angiogenesis involve measuring neovessel density in a manual fashion from fluorescence images. In another aspect, in order to obtain a more rapid, accurate assessment of angiogenesis, a Vascular Assessment and Measurement software (VAM) has been developed that utilizes artificial intelligence and machine learning (AI/ML) to identify and provide morphometric data from phase and fluorescence images of MV cultures. The VAM software is trained to recognize parent MV, neovessels, and non-MV artifacts. This analysis software functions coordinately with the Cytiva (formerly GE Healthcare) INCELL 6500 confocal scanning platform routinely used in the lab. In some aspects, visualization can be also achieved through antibody and/or fluorophore labeling.

In some aspects, the cells from the 3D culture can be assayed for varying levels of gene expression, enzymatic activity, and the like to assess for angiogenic effects, as well as through visualization and measurement of angiogenesis. Such additional steps are known and may include polymerase chain reactions, RNA isolation, DNA isolation, western blotting, Southern blotting, northern blotting, HPLC-MS/MS, MALDI-TOF, phenotypic screening, nucleic acid and/or protein sequencing, kinase assays, ELISA, electrophoresis, chromatography, flow cytometry and the like. In some aspects, visualization can be achieved through antibody and/or fluorophore labeling. In certain aspects, proteins and/or genes may present as markers of an agent's effect on angiogenesis.

### Examples

Angiomics™ Isolated, Human Microvessel Fragments.

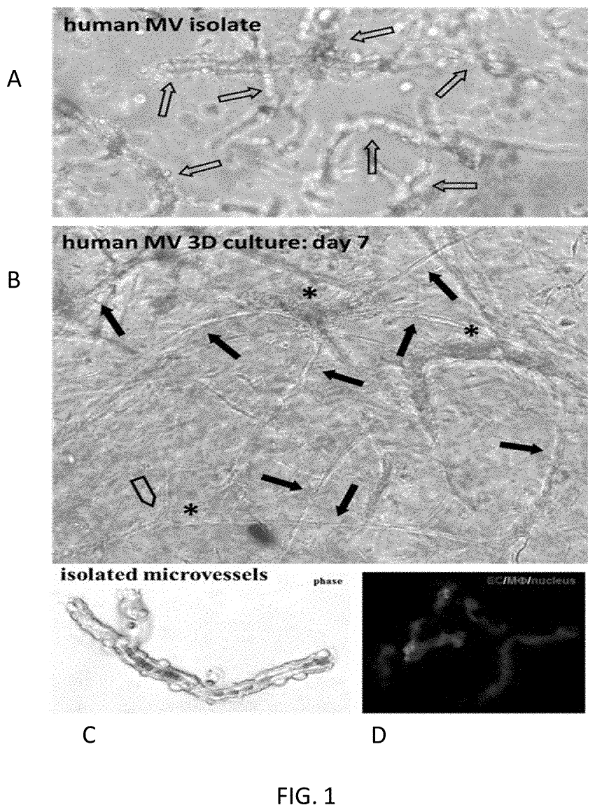

Because angiogenesis, vascular remodeling, and vascular stability depend not only on the endothelial cell, but also proper vessel architecture, mature matrix elements, and a spectrum of perivascular cells, a vascularization technology has been developed that utilizes freshly isolated microvessel fragments from adipose (FIG. 1). Importantly, these isolated microvessel fragments contain all vascular cells types, maintained in the native microvessel structure. When the constructs are placed in 3D matrix cultures, the individual microvessel fragments spontaneously sprout and grow, forming neovessels which will eventually fill the collagen gel (FIG. 1). When implanted as part of a tissue construct, the microvessel fragments recapitulate tissue neovascularization and form stable, perfused, hierarchical microvascular networks. Additionally, given the rapid means of vascularization, the isolated microvessel fragments have been explored in pre-clinical studies of tissue implants.

Neovascular Network Formation In Vitro.

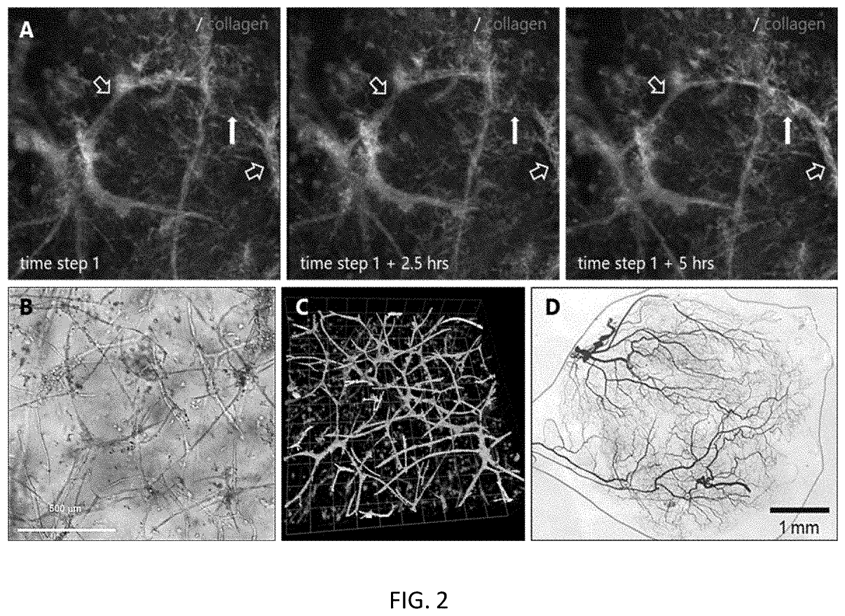

It has also been demonstrated that derivation and expansion of a neovasculature from isolated microvessel fragments in a stromal environment (i.e. made of collagen) in vitro enables rapid (within 24 hrs of transplantation) integration with the host circulation upon implantation. This is true for microvessel fragments derived from mouse, rat, and human. An important aspect of this dynamic is the ability for growing neovessels, as in the body, to locate and inosculate with each other creating a network of immature neovessels that fills the tissue space (FIG. 2). Because this network is interconnected while undergoing active angiogenesis, it can quickly locate and inosculate with an adjacent circulation and begin distributing blood throughout the neovascular work in the implanted graft. This intravascular perfusion then drives development of the fully functioning microcirculation. Interestingly, we have recently shown that stromal cells are important in guiding neovessels across tissue boundaries such as that present between a graft and the implant tissue.

Angiogenic Outgrowth from Organoids.

Conditions that promote angiogenesis from pre-vascularized organoids embedded in a 3D collagen environment have been developed. Using human MSCs, undifferentiated or differentiated, we found that 1) that there is an optimum ratio of the number of spheroid cells (MSCs in this example) to isolated human microvessel fragments to effectively vascularize the organoid and 2) this ratio impacts the degree of angiogenesis from this intra-organoid vasculature into a surrounding stromal environment. Importantly, because the outgrowing neovessels of the organoids are derived from the same parent microvessel fragments used to create a stromal neovascular network, the two should readily locate and inosculate with each other as we have shown in different applications.

Vascularized In Vitro Perfusion Module (VIPM™) (Perfusion Model).

Towards recapitulating the formation of a mature microcirculation in vitro, a solution has been developed whereby neovasculatures grown in 3D matrices from isolated, human microvessel fragments are integrated with fluidic channels connected to external flow pumps. The key elements to this approach involve fluidic channels (alone or lined with endothelial cells), growing the network of neovessels, inosculating neovessels of that network to the channels such that lumen are contiguous, and providing appropriate hemodynamic cues to drive intravascular flow through the neovascular network. The entire system is established in custom-made devices (made, for example, via 3D printing) that enables porting to and from the channels, channel formation, and long-term (weeks) culture of the neovasculatures.

To recapitulate the vascularization capabilities of the isolated microvessel fragments in vivo to derive a microcirculation in vitro, a fluidic-based approach that involves two parallel channels for inosculation by the isolated microvessel fragments and perfusion of the thereby derived neovasculature to drive neovascular remodeling was developed. The strategy entailed creating two parallel fluid flow channels (200-500 μm OD) 5 mm apart in a device made of 3D printed PDMS with wells constituting inlet and outlet media reservoirs. Media delivery and withdrawal to and from the device occurs via syringe pumps filling and emptying the reservoirs. The fluid column heights in these reservoirs establish defined hydrostatic pressures. The channels connect the different fluid reservoirs and extend through a collagen I-based tissue bed containing the isolated microvessel fragments. The PDMS device containing the collagen/microvessel bed with channeling constitutes the vascularized in vitro perfusion module (VIPM™).

To recapitulate tissue vascularization in the VIPM™ devices, a 3-stage process was developed and utilized beginning with isolated microvessel fragments seeded between the two channels to allow sprouting and early neovessel elongation from the parent isolated microvessel fragments during a non-perfused, static phase (i.e. no channel flow). Once angiogenesis was clearly occurring (after neovessel sprouting), media was perfused into and withdrawn from the inlet and outlet reservoirs, respectively, at 20 μl/hr during an interstitial flow-conditioning phase. In this configuration, media traverses the inlet channel to a shared reservoir and withdrawn back out via the outlet channel. At this flow rate, fluid flowed into the collagen interstitium from the inlet channel and out of the interstitium into the outlet channel.

During this interstitial flow-conditioning phase, neovascularity expanded within the collagen tissue space, with neovessels growing towards both channels while also inosculating with each other to form interconnected networks. During this time, neovessels approached and spontaneously inosculated with the walls of the two channels, with more inosculation events occurring at the outlet channel than at the inlet channel. Similar to the static angiogenesis phase, neovessels growing in the presence of interstitial flow were of uniform size and lacked significant perivascular cell coverage, similar to what is observed early following implantation prior to intravascular perfusion.

After inosculation, visible by phase imaging and typically occurring 3-5 days of interstitial flow conditioning, the exit end of the inlet channel was blocked by filling the associated reservoir with collagen while maintaining filling of the inlet reservoir with media. This resulted in the development of 1 cm of water (˜1 mm Hg) pre-load pressure to the inlet channel accompanying continued media flow through the channels. After blocking the channel with collagen, endothelial cells were delivered to the channel interior to line the channel walls prior to re-initiating flow to reduce the extent of fluid flow through the interstitium of the collagen. Indeed, fluid flow simulations indicates that, despite the increased pressure, interstitial flow fields remained unaffected. In this pressure conditioning phase, neovessel morphology and network topology began to change. The density of vessels in the network decreased, with the most pronounced drop in vessel numbers occurring at the inflow side of the network. Furthermore, the distribution of vessel diameters shifted from predominately small caliber vessels during the angiogenesis phases to a broader distribution of diameters including larger caliber vessels. A hierarchical organization of vessels across the network evolved between the two channels. Additionally, vessels at the inflow side of the network were less branched, larger in diameter, and associated with a greater perivascular cell coverage reminiscent of arterioles. Consistent with a more capillary-like appearance, vessels in the interior of the network were more numerous, branched, and smaller in caliber. At the outflow end of the network, vessel architecture was similar to the inflow end except that there were fewer vessel numbers, with a reduced perivascular cell coverage consistent with a venule-like morphology. These morphology changes associated with the pressure phase were accompanied by the progressive accumulation of a-actin-positive cells along the vessels and changes in the expression of genes related to microvessel maturation. Comparing the relative expression levels of the COL4A4 and LAMA4, gene products that are components of mature basement membranes, between harvested microvessel fragments (considered mature), and microvessel fragments in the static angiogenesis phase, the interstitial flow phase, and the pressure phase indicates these genes are down regulated during the two angiogenic/inosculation phases and return to similar or higher levels of expression during the pressure phase as the mature isolate. Additional genes such as ADORA2A, expressed by endothelial cells promotes vasodilation in the vasculature, and CSPG4 (or NG2), a marker of differentiated perivascular cells, were similarly regulated. These morphological and gene expression changes consistent with microvascular maturation were concomitant with intravascular access of fluorescent beads introduced into the media via the inlet channels. Video recordings indicate that the beads (1 μm OD) moved along vessel paths of the vascular network. Confocal imaging of fixed microvasculatures revealed the presence of beads, introduced via the inlet channel, lodged within patent lumens of microvessel fragments of the network, indicating intravascular perfusion of the network.

Fluidics channels, arranged in a device enabling control of fluid perfusion and channel-vessel interactions, serve as the avenues for the hemodynamic cues driving adaptation and remodeling of the neovascular network. This approach resulted in the formation of an in vitro microcirculation that exhibited 1) a perfused, hierarchical network of microvessel fragments, 2) a broader distribution of vessel diameters, 3) perivascular cell dynamics consistent with vascularization processes, 4) and gene expression changes consistent with angiogenesis and vessel maturation. Progressive changes to vessel segment morphology and character associated with a maturing neovasculature in the microvasculatures of this system were observed. During angiogenesis, neovessel calibers remained small until the pressure conditioning phase wherein diameters become more distributed due to larger caliber vessels developing. Similarly, perivascular cell coverage, which is reduced during angiogenesis, concomitantly increased in the pressure phase reflecting neovessel maturation. Finally, genes expressed in endothelial and perivascular cells of mature microvasculatures were down-regulated during angiogenesis and subsequently upregulated during the pressure phase. All these observations are consistent with the transition from angiogenesis to a mature microcirculation during the pressure phase in the VIPM™.

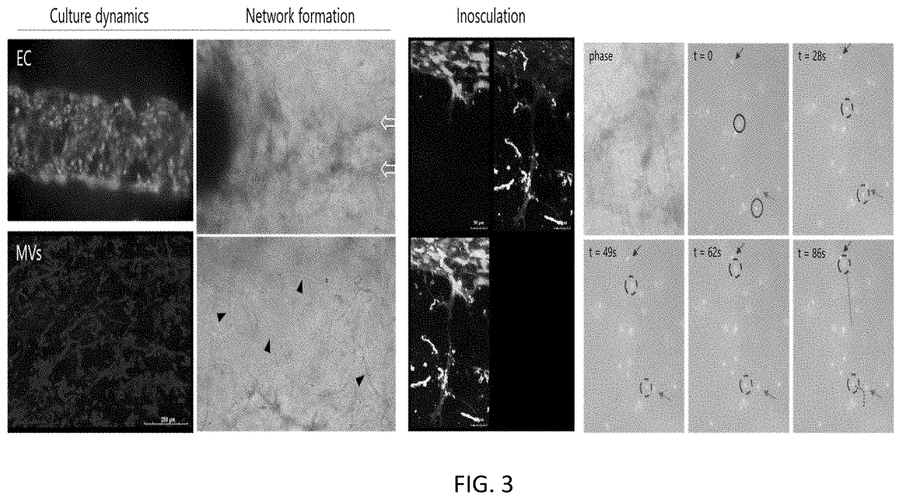

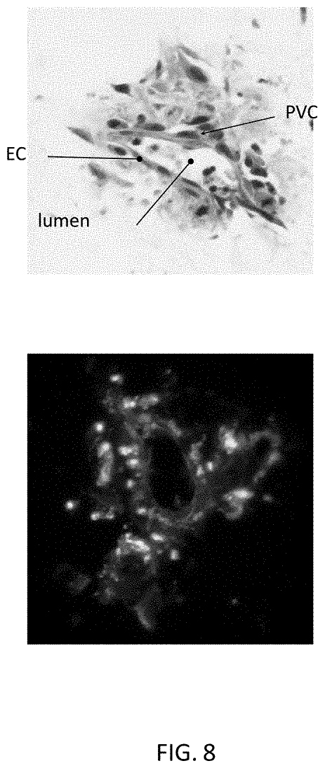

A critical aspect to establishing the in vitro microcirculation was the staging of interstitial flows and pressures. Establishment of the microcirculation involved 3 general phases beginning with the induction of angiogenesis under no channel flow conditions (static phase) followed by introduction of fluid flow through the channels and into the interstitium of the vascularized collagen bed (interstitial flow phase) followed by pressurizing the inlet channel (pressure conditioning phase). Similar to what was observed in constructs established in standard well plates, seeded parent microvessel fragments undergo angiogenesis in the absence of channel flow (i.e. under static conditions) in the VIPM™. During this static phase, neovessels sprouted and began to elongate. Establishing channel, and therefore interstitial, flow enhanced angiogenesis and promoted neovascular networking. Interestingly, it was important to begin the interstitial flow phase after neovessels began to elongate. In this regard, interstitial flow in the VIPM™ suppressed angiogenic sprouting and only after the neovessel began to grow did interstitial flow promote angiogenesis. This differs from studies in which positive effects of interstitial flow on endothelial cell-based vasculogenesis and sprouting are described. Neovessel sprouting from the parent isolated microvessel fragments necessarily involves loosening of the microvessel wall to enable sprout formation. Perhaps, interstitial flow stabilizes the microvessel structure preventing sprout initiation even in the presence of an angiogenesis stimulator such as VEGF. While interstitial flows enhanced angiogenesis (once sprouting started) in the VIPM™, the extent of angiogenesis (determined by vessel length densities) was independent of the interstitial flow direction as similar densities occurred whether the interstitial flow was in the direction of fluid leaving the channel and entering the collagen tissue space (i.e., the inlet channel) or fluid moved from the collagen tissue space into the channel (i.e., the outlet channel). However, while neovessels actively approached and inosculated with the outlet channel walls during this interstitial flow phase, they did so at reduced levels at the inlet channel. This resulted in fewer inosculation events and a less stable inosculation structure as those that did form disassembled once the channel was pressurized. In a different configuration, instead of pressurizing the inlet channel, the outlet channel was pressurized, reasoning that the outlet channel had more, stable inosculation events. FIG. 8 sets forth both H&E and fluorescent images of the formed microvasculature. FIG. 3 depicts example images of an endothelial cell (EC)-lined channel surrounded by growing neovessels forming a network (black arrow heads in phase image) adjacent to the channel walls (open arrows). Neovessels inosculate with the ECs of the channel enabling perfusion of beads (right panels) as shown by still images from real-time video showing two beads moving through neovessels (upper left). Dashed lines indicate flow paths. Stationary beads are marked for positional reference.

Tumor-VIPM™.

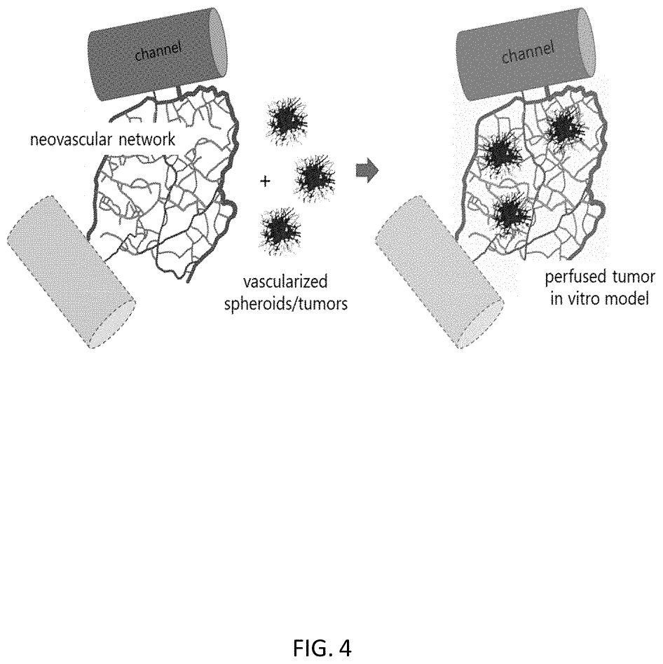

To build a vascular-perfused tumor model, pre-vascularized tumor spheroids or tumor fragments are integrated within a 3D bed of angiogenic neovessels in a way that promotes angiogenesis from the spheroid leading to inosculation of spheroid vessels with the surrounding stromal vessels (FIG. 4).

The general strategy for making perfused tumor models is to leverage these dynamics and capabilities to combine variations of tumor cells, including pre-vascularized, tumor organoids with a pre-vascularized stromal space to model the vascular-stromal-tumor compartments.

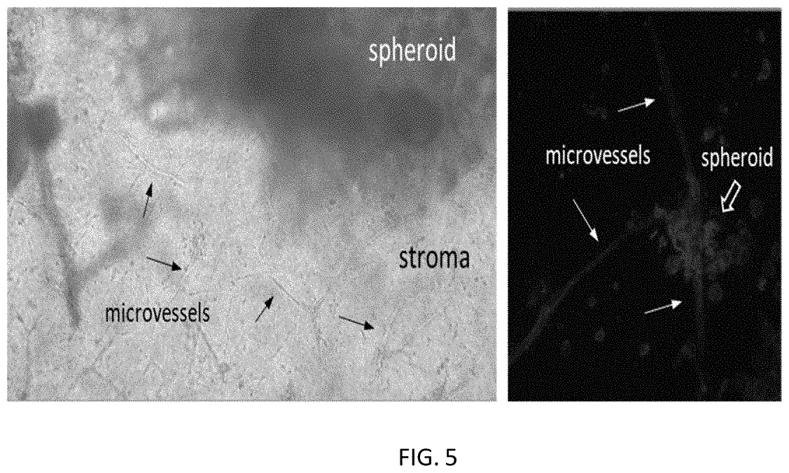

The configuration in which the tumor cells and/or organoids are integrated into the vascularized stroma of the VIPM can vary depending on the application. For example, two configurations possible configurations involve creating models of epithelial-mesenchymal transformation and metastasis. In Model 1, an epithelial tissue-stroma interface is created in which pre-cancerous or neoplastic cells are cultured on top of a 3D collagen matrix coated with relevant basement membrane proteins to establish the tissue interface such as exists in the gut mucosa or breast ducts. A perfused microcirculation sits subjacent to the epithelial interface (FIG. 5). In a second configuration, tumor organoids comprised of tumor cells and isolated microvessel fragments are cultured in the presence of the forming neovasculature during which the growing neovessels of the organoid and the neovasculature locate and inosculate with each other. The perfusion model is subsequently developed with contiguous perfusion of the tumor organoids (FIG. 5). While this configuration enables modeling metastasis by examining tumor cell intravasation (and possible extravasation in a 2nd downstream perfusion model), this configuration also models more native-like tumor biology facilitating screening, therapeutic investigations, immune cell-tumor interactions, etc.

Tumor Spheroid—Neovascular Interactions.

A key step in integrating pre-vascularized tumor spheroids into the VIPM microcirculation requires an angiogenic neovasculature to merge with the tumor spheroid. Towards this end, tumor organoids comprised of MCF-7 breast cancer cells and human, isolated microvessel fragments are created. After a short culture period (1-3 days) to form the organoid, the pre-vascularized tumor organoids are combined with microvessel fragments and stromal matrix (collagen I) to form a tissue construct. In these experiments, these constructs did not contain the channels of the perfusion model. In this setting, neovessels grow toward and directly interact with the tumor organoids (FIG. 9).

The VIPM-tumor model creates a vascularized tissue bed containing a perfused tumor organoid. The disclosed tumor models allow for studies that were previously not possible, including but not limited to investigating immune cell homing to tumors, drug delivery mechanisms and avenues, primary tumor cell escape processes, tumor-stromal cell interactions, tumor-tissue dynamics, tumor-vascular dynamics, etc. These studies can all involve tumor cell lines and/or primary tumor cells.

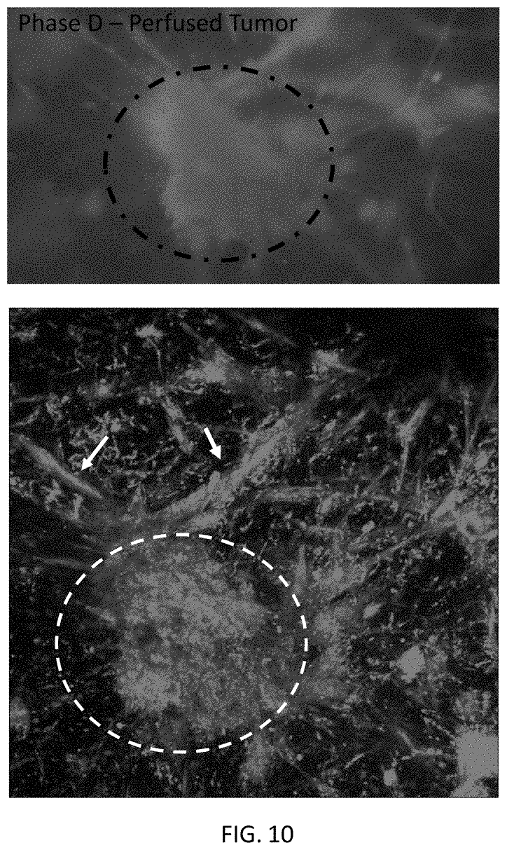

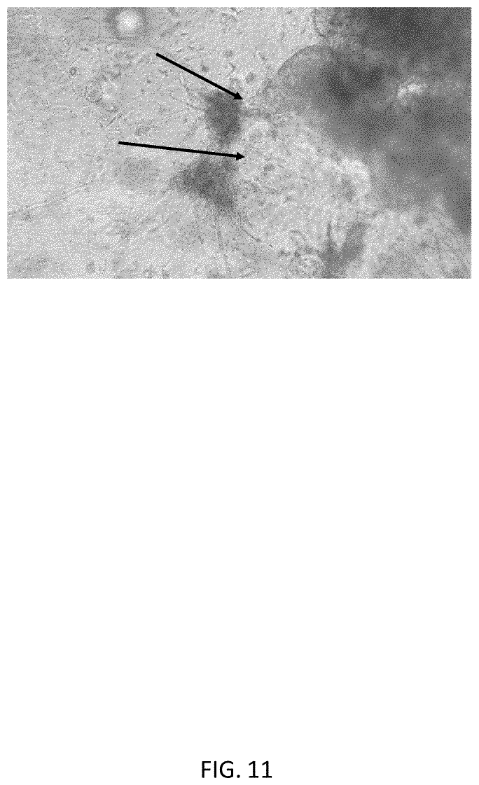

Images of tumor cells, as spheroids, integrated in the vascularized tissue space confirm the interaction of vessels with the tumor spheroid. FIG. 10 sets forth a confocal microscopy image with clear interfacing of the vasculature with the bulk of the tumor spheroid. FIG. 11 sets forth a phase microscopy image of a spheroid similarly interacting with the microvasculature.

Further, by combining tumor organoids (vascularized tumor spheroids) with other tissue organoids, the disclosed model enables in vitro studies of tumor metastasis. In one example, the other tissue organoids may include a lymph node, lung organoid, liver organoid, brain organoid, bone organoid, and the like. Tissue organoid selection may be based on a tissue that the particular tumor type typically disseminates to during metastasis (e.g., breast cancer disseminating to adjacent lymph nodes). Accordingly, the presently disclosed models have application in studying metastasis of tumors.

A first aspect of the present disclosure, either alone or in combination with any other aspect, concerns a three-dimensional (3D) tumor model comprising: tumor cells; and isolated microvessel fragments or a microvasculature developed therefrom, wherein the isolated microvessel fragments or the microvasculature are embedded within a polymerized medium comprised of extracellular matrix.

A second aspect of the present disclosure, either alone or in combination with any other aspect, concerns the 3D tumor model of the first aspect, wherein the extracellular matrix comprises collagen, fibrin, Matrigel, laminin.

A third aspect of the present disclosure, either alone or in combination with any other aspect, concerns the 3D tumor model of the first aspect, wherein the tumor cells are part of a tumor organoid, a tumor spheroid, or a pre-vascularized tumor fragment.

A fourth aspect of the present disclosure, either alone or in combination with any other aspect, concerns the 3D tumor model of the first aspect, further comprising: a first and a second channel, wherein the two channels are parallel and wherein the first and second channels are embedded within the polymerized medium, and wherein the isolated microvessel fragments or the microvasculature developed therefrom are in a space between the first and second channels.

A fifth aspect of the present disclosure, either alone or in combination with any other aspect, concerns the 3D tumor model of the third aspect, wherein each of the first and second channels comprises an inlet end and an outlet end and further wherein a fluid source is operably connected to each inlet end.

A sixth aspect of the present disclosure, either alone or in combination with any other aspect, concerns the 3D tumor model of the fifth aspect, wherein each outlet end is operably connected to an outlet reservoir.

A seventh aspect of the present disclosure, either alone or in combination with any other aspect, concerns the 3D tumor model of the sixth aspect, further comprising an at least partial obstruction at the outlet end of the first channel to provide a pre-load pressure.

An eighth aspect of the present disclosure, either alone or in combination with any other aspect, concerns the 3D tumor model of the seventh aspect, wherein the at least partial obstruction comprises a collagen plug.

A ninth aspect of the present disclosure, either alone or in combination with any other aspect, concerns the 3D tumor model of the sixth aspect, wherein the outlet reservoir is operably connected to at least the inlet end of the second channel.

A tenth aspect of the present disclosure, either alone or in combination with any other aspect, concerns the 3D tumor model of the first aspect, wherein one or more extracellular matrix proteins and/or structures are in contact with the tumor cells.

An eleventh aspect of the present disclosure, either alone or in combination with any other aspect, concerns the 3D tumor model of the tenth aspect, wherein the one or more extracellular matrix proteins and/or structures comprise basement membrane proteins and/or structures.

A twelfth aspect of the present disclosure, either alone or in combination with any other aspect, concerns a method for preparing a vascularized 3D tumor model comprising: providing isolated microvessel fragments to a space between two channels embedded within a polymerized medium; and providing tumor cells on or embedded within the polymerized medium.

A thirteenth aspect of the present disclosure, either alone or in combination with any other aspect, concerns the method of the twelfth aspect, wherein the tumor cells are part of a tumor organoid, a tumor spheroid, or a pre-vascularized tumor fragment.

A fourteenth aspect of the present disclosure, either alone or in combination with any other aspect, concerns the method of the twelfth aspect, wherein a fluid media is perfused through the inlet of one channel to an outlet reservoir and back through an inlet of the second channel.

A fifteenth aspect of the present disclosure, either alone or in combination with any other aspect, concerns the method of the fourteenth aspect, wherein the fluid media is perfused at a rate of about 20 μL/hour.

A sixteenth aspect of the present disclosure, either alone or in combination with any other aspect, concerns the method of the twelfth aspect, wherein the tumor cells are in contact with a one or more extracellular matrix proteins and/or structures.

A seventeenth aspect of the present disclosure, either alone or in combination with any other aspect, concerns the method of the twelfth aspect, further comprising providing isolated endothelial cells to the fluid media.

An eighteenth aspect of the present disclosure, either alone or in combination with any other aspect, concerns the method of the seventeenth aspect, wherein at least one outlet end is at least partially obscured to create a pre-load pressure in the channel.

A nineteenth aspect of the present disclosure, either alone or in combination with any other aspect, concerns the method of the eighteenth aspect, wherein a collagen plug is used to at least partially obscure the at least one outlet end.

A twentieth aspect of the present disclosure, either alone or in combination with any other aspect, concerns the method of the twelfth aspect, wherein at least one outlet end is at least partially obscured to create a pre-load pressure in the channel.

A twenty-first aspect of the present disclosure, either alone or in combination with any other aspect, concerns a method for preparing a vascularized 3D tumor model comprising: providing isolated microvessel fragments to a space between a first channel and a second channel embedded within a polymerized medium; incubating the isolated microvessel fragments within the polymerized medium for a period of four to six days or until angiogenesis is observed; perfusing a fluid media from an inlet reservoir through the first channel to an outlet reservoir, wherein the outlet reservoir is operably connected to the second channel such that the perfused fluid media can traverse the second channel, wherein the fluid media is perfused for about three to five days or until the isolated microvessel fragments have visibly inosculated; at least partially obscuring the first channel at the outlet to the outlet reservoir to provide an increased pre-load pressure; re-initiating perfusion of the fluid media; and providing tumor cells on or embedded within the polymerized medium.

A twenty-second aspect of the present disclosure, either alone or in combination with any other aspect, concerns the method of the twenty-first aspect, further comprising providing isolated endothelial cells to at least the first channel.

A twenty-third aspect of the present disclosure, either alone or in combination with any other aspect, concerns the method of the twenty-first aspect, wherein the tumor cells are provided prior to incubation of the isolated microvessel fragments.

A twenty-fourth aspect of the present disclosure, either alone or in combination with any other aspect, concerns the method of the twenty-first aspect, wherein the tumor cells are provided following the re-initiation of perfusion of the fluid media.

A twenty-fifth aspect of the present disclosure, either alone or in combination with any other aspect, concerns the method of the twenty-first aspect, wherein the fluid media is perfused at a rate of about 10 to 1000 μL/hr.

A twenty-sixth aspect of the present disclosure, either alone or in combination with any other aspect, concerns the method of the twenty-first aspect, wherein the increased pre-load pressure is of about 0.5 mm of Hg to 100 mm of Hg.

A twenty-seventh aspect of the present disclosure, either alone or in combination with any other aspect, concerns a method for preparing a vascularized 3D model comprising: providing isolated microvessel fragments to a space between a first channel and a second channel embedded within a polymerized medium; incubating the isolated microvessel fragments within the polymerized medium for a period of four to six days or until angiogenesis is observed; perfusing a fluid media from an inlet reservoir through the first channel to an outlet reservoir, wherein the outlet reservoir is operably connected to the second channel such that the perfused fluid media can traverse the second channel, wherein the fluid media is perfused for about three to five days or until the isolated microvessel fragments have visibly inosculated; at least partially obscuring the first channel at the outlet to the outlet reservoir to provide an increased pressure; providing isolated endothelial cells to the first and second channels; and re-initiating perfusion of the fluid media.

A twenty-eighth aspect of the present disclosure, either alone or in combination with any other aspect, concerns the method of the twenty-seventh aspect, further comprising providing isolated endothelial cells to at least the first channel.

A twenty-ninth aspect of the present disclosure, either alone or in combination with any other aspect, concerns the method of the twenty-seventh aspect, wherein the tumor cells are provided prior to incubation of the isolated microvessel fragments.

A thirtieth aspect of the present disclosure, either alone or in combination with any other aspect, concerns the method of the twenty-seventh aspect, wherein the tumor cells are provided following the re-initiation of perfusion of the fluid media.

A thirty-first aspect of the present disclosure, either alone or in combination with any other aspect, concerns the method of the twenty-seventh aspect, wherein the fluid media is perfused at a rate of about 10 to 1000 μL/hr.

A thirty-second aspect of the present disclosure, either alone or in combination with any other aspect, concerns the method of the twenty-seventh aspect, wherein the increased pre-load pressure is of about 0.5 mm of Hg to 100 mm of Hg.

A thirty-third aspect of the present disclosure, either alone or in combination with any other aspect, concerns a 3D angiogenesis model comprising isolated microvessel fragments or a microvasculature developed therefrom between two parallel channels embedded within a polymerized medium, wherein each channel comprises an inlet end and an outlet end, each inlet end being operably connected to a fluid media source and wherein at least one outlet end is operably linked to the inlet end of a different channel and wherein the fluid media is actively pumped into at least one channel to allow for interstitial flow-conditioning.

A thirty-fourth aspect of the present disclosure, either alone or in combination with any other aspect, concerns the 3D angiogenesis model of the thirty-third aspect, further comprising tumor cells on or embedded within the polymerized medium.

A thirty-fifth aspect of the present disclosure, either alone or in combination with any other aspect, concerns the 3D angiogenesis model of the thirty-third aspect, further comprising an at least partial obstruction at the outlet end of the first channel to provide a pre-load pressure.

A thirty-sixth aspect of the present disclosure, either alone or in combination with any other aspect, concerns the 3D angiogenesis model of the thirty-fifth aspect, wherein the at least partial obstruction comprises a collagen plug.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified. Methods of nucleotide amplification, cell transfection, and protein expression and purification are similarly within the level of skill in the art.

Patents, publications, and applications mentioned in the specification are indicative of the levels of those skilled in the art to which the invention pertains. These patents, publications, and applications are incorporated herein by reference to the same extent as if each individual patent, publication, or application was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

We claim:

1. A three-dimensional (3D) tumor model comprising:
tumor cells; and,
isolated microvessel fragments or a microvasculature developed therefrom,
wherein the isolated microvessel fragments or the microvasculature are embedded within a polymerized medium comprised of extracellular matrix.

2. The 3D tumor model of claim 1, wherein the extracellular matrix comprises at least one of collagen I, collagen II, collagen III, collagen IV, fibrin, Matrigel, laminin, nidogen, perlecan sulfated glycolipids, glycoproteins and proteoglycans.

3. The 3D tumor model of claim 1, wherein the tumor cells are alone or part of a tumor organoid, a tumor spheroid, or a pre-vascularized tumor fragment.

4. The 3D tumor model of claim 1, further comprising:
a first and a second channel, wherein the two channels are parallel and wherein the first and second channels are embedded within the polymerized medium, and wherein the isolated microvessel fragments or the microvasculature developed therefrom are in a space between the first and second channels.

5. The 3D tumor model of claim 3, wherein each of the first and second channels comprises an inlet end and an outlet end and further wherein a fluid source is operably connected to each inlet end.

6. The 3D tumor model of claim 5, wherein each outlet end is operably connected to an outlet reservoir.

7. The 3D tumor model of claim 6, further comprising an at least partial obstruction at the outlet end of the first channel to provide a pre-load pressure.

8. The 3D tumor model of claim 7, wherein the at least partial obstruction comprises a collagen plug.

9. The 3D tumor model of claim 6, wherein the outlet reservoir is operably connected to at least the inlet end of the second channel.

10. The 3D tumor model of claim 1, wherein one or more extracellular matrix proteins and/or structures are in contact with the tumor cells.

11. The 3D tumor model of claim 10, wherein the one or more extracellular matrix proteins and/or structures comprise basement membrane proteins and/or structures.

12. A method for preparing a vascularized 3D tumor model comprising:
providing isolated microvessel fragments to a space between two channels embedded within a polymerized medium; and
providing tumor cells on or embedded within the polymerized medium.

13. The method of claim 12, wherein the tumor cells are part of a tumor organoid, a tumor spheroid, or a pre-vascularized tumor fragment.

14. The method of claim 12, wherein a fluid media is perfused through the inlet of one channel to an outlet reservoir and back through an inlet of the second channel.

15. The method of claim 14, wherein the fluid media is perfused at a rate of about 10=−5000 μL/hour.

16. The method of claim 12, wherein the tumor cells are in contact with a one or more extracellular matrix proteins and/or structures.

17. The method of claim 12, further comprising providing isolated endothelial cells to the fluid media.

18. The method of claim 17, wherein at least one outlet end is at least partially obscured to create a pre-load pressure in the channel.

19. The method of claim 18, wherein a collagen plug is used to at least partially obscure the at least one outlet end.

20. The method of claim 12, wherein at least one outlet end is at least partially obscured to create a pre-load pressure in the channel.

21. A method for preparing a vascularized 3D tumor model comprising:
providing isolated microvessel fragments to a space between a first channel and a second channel embedded within a polymerized medium;
incubating the isolated microvessel fragments within the polymerized medium for a period until angiogenesis is observed;
perfusing a fluid media from an inlet reservoir through the first channel to an outlet reservoir, wherein the outlet reservoir is operably connected to the second channel such that the perfused fluid media can traverse the second channel, wherein the fluid media is perfused for about three to five days or until the isolated microvessel fragments have visibly inosculated;
at least partially obscuring the first channel at the outlet to the outlet reservoir to provide an increased pre-load pressure;
re-initiating perfusion of the fluid media; and
providing tumor cells on or embedded within the polymerized medium.

22. The method of claim 21, further comprising providing isolated endothelial cells to at least the first channel.

23. The method of claim 21, wherein the tumor cells are provided prior to incubation of the isolated microvessel fragments.

24. The method of claim 21, wherein the tumor cells are provided following the re-initiation of perfusion of the fluid media.

25. The method of claim 21, wherein the fluid media is perfused at a rate of about 10 to 5000 μL/hr.

26. The method of claim 21, wherein the increased pre-load pressure is of about 0.5 mm of Hg to 160 mm of Hg.

27. A method for preparing a vascularized 3D model comprising:
providing isolated microvessel fragments to a space between a first channel and a second channel embedded within a polymerized medium;
incubating the isolated microvessel fragments within the polymerized medium for a period until angiogenesis is observed;
perfusing a fluid media from an inlet reservoir through the first channel to an outlet reservoir, wherein the outlet reservoir is operably connected to the second channel such that the perfused fluid media can traverse the second channel, wherein the fluid media is perfused for about three to five days or until the isolated microvessel fragments have visibly inosculated;
at least partially obscuring the first channel at the outlet to the outlet reservoir to increase channel preload;
providing isolated endothelial cells to the first and/or second channels; and
re-initiating perfusion of the fluid media.

28. The method of claim 27, further comprising providing isolated endothelial cells to at least the first channel.

29. The method of claim 27, wherein the tumor cells are provided prior to incubation of the isolated microvessel fragments.

30. The method of claim 27, wherein the tumor cells are provided following the re-initiation of perfusion of the fluid media.

31. The method of claim 27, wherein the fluid media is perfused at a rate of about 10 to 5000 μL/hr.

32. The method of claim 27, wherein the increased pre-load pressure is of about 0.5 mm of Hg to 160 mm of Hg.

33. A 3D angiogenesis model comprising isolated microvessel fragments or a microvasculature developed therefrom between two parallel channels embedded within a polymerized medium, wherein each channel comprises an inlet end and an outlet end, each inlet end being operably connected to a fluid media source and wherein at least one outlet end is operably linked to the inlet end of a different channel and wherein the fluid media is actively pumped into at least one channel to allow for interstitial flow-conditioning.

34. The 3D angiogenesis model of claim 33, further comprising tumor cells on or embedded within the polymerized medium.

35. The 3D angiogenesis model of claim 33, further comprising an at least partial obstruction at the outlet end of the first channel to provide a pre-load pressure.

36. The 3D angiogenesis model of claim 35, wherein the at least partial obstruction comprises a collagen plug.
